(19) United States
(12) Patent Application Publication
(10) Pub. No.: US 2026/0263240 A1
(43) Pub. Date: Sep. 10, 2026

(54) IMPLANTABLE MEDICAL DEVICE WITH A CONDUCTIVE LAYER HAVING A BONDING RIM FOR COUPLING TO A FLUIDIC MANIFOLD

(71) Applicant: Boston Scientific Scimed, Inc., Maple Grove, MN (US)

(72) Inventors: Eduardo Marcos Larangeira, Cork City (IE); Thomas Sinnott, Enniscorthy (IE); Laurence Norris, Clonmel (IE); Kevin P. McCarthy, Waterford (IE)

(21) Appl. No.: 19/549,658

(22) Filed: Feb. 25, 2026

Related U.S. Application Data
(60) Provisional application No. 63/766,707, filed on Mar. 4, 2025.

(51) Int. Cl. A61F 2/48; A61F 2/00; A61F 2/26

(52) U.S. Cl. CPC A61F 2/482; A61F 2/004; A61F 2/26; A61F 2/484; A61F 2240/001; A61F 2250/0013

(57) ABSTRACT

An implantable medical device may include an inflatable member, a fluid reservoir, and an electronic pump device configured to transfer fluid between the inflatable member and the fluid reservoir. The electronic pump device includes a housing. The housing includes a fluidic manifold and a fluidic component coupled to the fluidic manifold. The fluidic component includes a conductive layer. The conductive layer includes a bonding rim. The bonding rim is coupled to the fluidic manifold.

### CROSS-REFERENCE TO RELATED APPLICATION

This application claims priority to U.S. Provisional Patent Application No. 63/766,707, filed on Mar. 4, 2025, entitled “IMPLANTABLE MEDICAL DEVICE WITH A CONDUCTIVE LAYER HAVING A BONDING RIM FOR COUPLING TO A FLUIDIC MANIFOLD”, the disclosure of which is incorporated by reference herein in its entirety.

### TECHNICAL FIELD

This disclosure relates generally to an implantable medical device with a conductive layer having a bonding rim configured to be coupled to a fluidic manifold.

### BACKGROUND

Some inflatable medical devices have a pump device, which, when operated by a user, causes a transfer of fluid between a fluid reservoir and an inflatable member. The pump device may have fluidic components (e.g., pump(s) and/or valves), where a fluidic component may have multiple components. Attaching a fluidic component to a housing of a pump device may cause one or more technical problems in terms of performance and/or reliability. In some examples, using heat to couple a fluidic component may cause deformation to the fluidic component itself and/or one or more components of the pump device.

### SUMMARY

This disclosure relates to an inflatable medical device with an electronic pump device, where the electronic pump device includes a fluidic manifold with one or more fluidic components such as pump(s), valve(s) and/or pressure sensor(s). A fluidic component includes a conductive layer. In some examples, the conductive layer includes a disc-shaped conductive element that is positioned into a recess of the fluidic manifold. In some examples, the conductive layer is a base plate of a valve or a pump. A base plate may define an inlet hole (e.g., a through hole) and an outlet hole (e.g., a through hole). In some examples, the base pate is a metal-based material. In some examples, the conductive layer is a passive valve layer defining an inlet port or an outlet port. In some examples, the conductive layer is one or more layers of a pressure sensor. The conductive layer includes a bonding rim that is coupled to the fluidic manifold or another layer of the fluidic component. In some examples, the bonding rim is a flap, a rim, or other surface feature of the conductive layer. In some examples, the conductive layer may be coupled to the fluidic manifold or the other layer of the fluidic component based on a fusion weld (e.g., applying heat to the two metal components). The bonding rim may reduce deformation to the fluidic component and/or the fluidic manifold. The bonding rim may be a portion (e.g., a perimeter or circumferential portion) of the conductive layer. In some examples, the conductive layer includes a channel disposed around the perimeter of the conductive layer, where the bonding rim is defined by the channel. In some examples, the bonding rim includes a raised portion (or an extended portion) that extends from a surface or an edge of the conductive layer.

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold; and a fluidic component coupled to the fluidic manifold, the fluidic component including a conductive layer, the conductive layer including a bonding rim, the bonding rim being coupled to the fluidic manifold.

In some aspects, the techniques described herein relate to an electronic pump device including: a fluidic manifold; and a fluidic component coupled to the fluidic manifold, the fluidic component including a conductive layer, the conductive layer including a bonding rim, the bonding rim being coupled to the fluidic manifold.

In some aspects, the techniques described herein relate to a method for constructing an electronic pump device, the method including: inserting at least a portion of a fluidic component into a recess of a fluidic manifold, the fluidic component including a conductive layer, the conductive layer including a bonding rim; and coupling the bonding rim to a portion of the fluidic manifold.

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold; and a fluidic component coupled to the fluidic manifold, the fluidic component including a conductive layer, the conductive layer including a bonding rim, the bonding rim being coupled to the fluidic manifold.

In some aspects, the techniques described herein relate to an electronic pump device including: a fluidic manifold; and a fluidic component coupled to the fluidic manifold, the fluidic component including a conductive layer, the conductive layer including a bonding rim, the bonding rim being coupled to the fluidic manifold.

In some aspects, the techniques described herein relate to a method for constructing an electronic pump device, the method including: inserting at least a portion of a fluidic component into a recess of a fluidic manifold, the fluidic component including a conductive layer, the conductive layer including a bonding rim; and coupling the bonding rim to a portion of the fluidic manifold.

### BRIEF DESCRIPTION OF THE DRAWINGS

FIG. 1 illustrates an implantable medical device with an electronic pump device having a fluidic component with a base plate according to an aspect.

FIG. 2A illustrates an example of a fluidic manifold according to an aspect.

FIG. 2B illustrates a circuit substrate coupled to the fluidic manifold according to an aspect.

FIG. 2C illustrates a perspective of the fluidic manifold according to an aspect.

FIG. 2D illustrates a perspective of the fluidic manifold according to another aspect.

FIG. 2E illustrates a perspective of a circuit substrate coupled to a fluidic manifold according to an aspect.

FIG. 3 illustrates an example of a pump according to an aspect.

FIG. 4 illustrates an example of a valve according to an aspect.

FIG. 5A illustrates an example of a fluidic component disposed within a recess of a fluidic manifold according to an aspect.

FIG. 5B illustrates the fluidic component being coupled to the fluidic manifold based on a fusion weld according to an aspect.

FIG. 6A illustrates a base plate according to an aspect.

FIG. 6B illustrates a base plate according to another aspect.

FIG. 6C illustrates the base plate coupled to a fluidic manifold according to an aspect.

FIG. 7A illustrates a base plate according to an aspect.

FIG. 7B illustrates a base plate according to another aspect.

FIG. 8A illustrates a base plate according to an aspect.

FIG. 8B illustrates a base plate according to another aspect.

FIG. 8C illustrates the base plate coupled to a fluidic manifold according to an aspect.

FIG. 9 illustrates a fluidic component being coupled to the fluidic manifold based on a fusion weld according to an aspect.

FIG. 10 illustrates an inflatable medical device according to an aspect.

FIG. 11 illustrates an inflatable medical device according to an aspect.

FIG. 12 illustrates an exploded view of a housing of an electronic pump device according to an aspect.

FIG. 13 illustrates a perspective of an inflatable penile prosthesis according to an aspect.

FIG. 14 illustrates an example of an artificial urinary sphincter device according to an aspect.

FIG. 15 illustrates a flowchart depicting example operations of coupling a fluidic component to a fluidic manifold of an electronic pump device according to an aspect.

### DETAILED DESCRIPTION

This disclosure relates to an implantable medical device with an electronic pump device configured to automatically transfer fluid between a fluid reservoir and an inflatable member. In some examples, the implantable medical device includes a penile prosthesis with one or more inflatable cylinders. In some examples, the implantable medical device includes a urinary control device with an inflatable cuff. However, the implantable medical device may include other types of medical devices such as a stimulation device or a neuromodulation device. The electronic pump device includes a housing with a fluidic manifold having a fluidic component to facilitate the transfer of fluid between the fluid reservoir and the inflatable member. In some examples, the fluidic component includes a pump (e.g., a piezo-electric pump). In some examples, the fluidic component includes a valve (e.g., a piezo-electric valve). In some examples, the fluidic component includes a pressure sensor.

A fluidic component includes a conductive layer (e.g., a circular or disc-shaped element). In some examples, the conductive layer is a base plate of a pump or a valve. A base plate includes a first hole (e.g., an inlet hole) and a second hole (e.g., an outlet hole). In some examples, the conductive layer is a passive valve layer defining an inlet valve or an outlet valve. The passive valve layer may include a helical slot configured to operate as a control valve element, and, in some examples, a hole through the passive valve layer. In some examples, the conductive layer is a layer of a pressure sensor. In some examples, the fluidic component includes multiple (stacked) components such as a plurality of circular (thin) elements. In some examples, the fluidic component is a pump or a valve, and the fluidic component includes actuator and a base plate, where the base plate is an example of the conductive layer. In some examples, the base plate is a component that is separate or distinct from the fluidic manifold and then coupled to the fluidic manifold based on heat welding. In some examples, the base plate is a component that is integral to the fluidic manifold, and another component (e.g., a passive valve layer, an actuator diaphragm, an isolation layer. etc.) is coupled to the base plate using heat welding.

The actuator of a pump or a valve may receive an electrical signal, in response to the electrical signal, the actuator may deform (e.g., bend, change its shape, etc.). The actuator may include one or more multiple components that are coupled together. In some examples, the actuator includes one or more disc-shaped elements. In some examples, the actuator includes an activation element (e.g., a piezo element with one or more electrodes, a piezo disc actuator, etc.) and an actuator diaphragm coupled to the activation element. The actuator diaphragm may be a flexible membrane. In some examples, the actuator diaphragm may be a metal-based flexible membrane. In some examples, the actuator includes a piezo disc actuator, an isolation layer, an adhesive material, and an actuator diaphragm. An electrical signal applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump or the valve.

The base plate may be a disc-shaped metal element. The base plate includes a first hole (e.g., a first through-hole) and a second hole (e.g., a second through-hole). In some examples, the first hole is an inlet hole, and the second hole is an outlet hole. Fluid may be transferred through the base plate via the first and/or second holes. In some examples, the first hole has a size (e.g., a diameter) that is different from the size (e.g., a diameter) of the second hole.

The conductive layer includes a bonding rim that is coupled to the fluidic manifold. The bonding rim may be a flap, a rim, or other surface feature configured to absorb heat and be coupled to the fluidic manifold or another component of the fluidic component. In some examples, the conductive layer may be coupled to the fluidic manifold or another component of the fluidic component based on a fusion weld (e.g., laser welding). The bonding rim may reduce deformation to the fluidic component and/or the fluidic manifold. The bonding rim may be a portion (e.g., a perimeter or circumferential portion) of the conductive layer. In some examples, the conductive layer includes a channel disposed around the perimeter of the conductive layer, where the bonding rim is defined by the channel. In some examples, the bonding rim includes a raised portion (or an extended portion) that extends from a surface or an edge of the conductive layer.

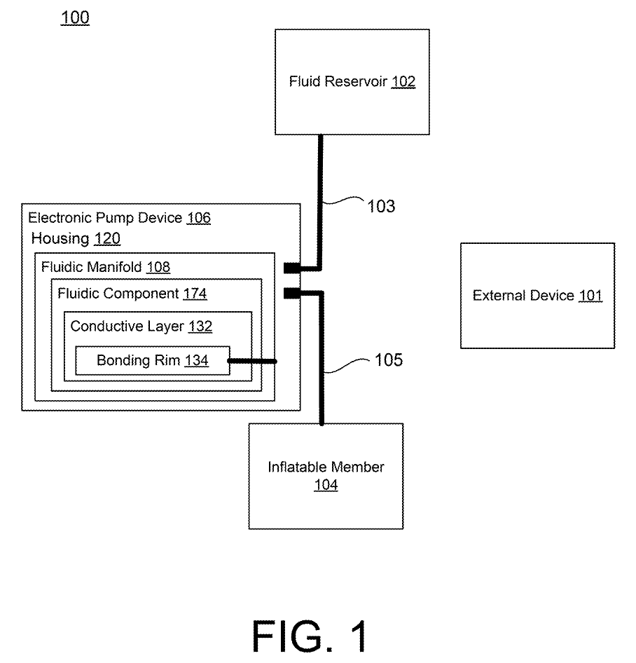

FIG. 1 illustrates an implantable medical device 100 according to an aspect. In some examples, the implantable medical device 100 is an artificial urinary sphincter device. In some examples, the implantable medical device 100 is an inflatable penile prosthesis. However, the implantable medical device 100 may include any type of medical device that transfers fluid between components of the implantable medical device 100 such as respiratory devices, cardiovascular devices, gastrointestinal devices, ophthalmic devices, drug delivery devices, and/or diagnostic devices. In some examples, the implantable medical device 100 may be a stimulation device. In some examples, the implantable medical device 100 may be a neuromodulation device.

The implantable medical device 100 includes a fluid reservoir 102, an inflatable member 104, and an electronic pump device 106 configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user’s abdominal cavity or the upper portion of the user’s pelvic cavity). In some examples, at least a portion of the electronic pump device 106 may be implemented in the patient’s body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

The electronic pump device 106 includes a housing 120. The housing 120 includes a fluidic manifold 108 that attaches fluid transfer and pressure regulating components. The fluidic manifold 108 may include a circuit substrate with electronic components. The fluidic manifold 108 includes one or more fluidic components 174 such as one or more pumps, one or more valves, and/or one or more pressure sensors. In some examples, a fluidic component 174 may include a plurality of disc-shaped members that are coupled together and coupled to the fluidic manifold. The type of individual elements that comprise a fluidic component 174 may depend on the type of fluidic component 174. In some examples, a fluidic component 174 includes a pressure sensor. In some examples, a fluidic component 174 includes a pump (e.g., a piezo-electric pump). In some examples, a fluidic component 174 includes a valve (e.g., a piezo-electric valve). A fluidic component 174 may include multiple components that are coupled to together (e.g., heat welding, one or more adhesive layers, male/female interlocking features, and/or fasteners, etc.).

The fluidic component 174 includes a conductive layer 132. The conductive layer 132 may be any type of element within a fluidic component 174 such as a pump, valve, or a pressure sensor. In some examples, the conductive layer 132 is a circular or a disc-shape element. In some examples, the conductive layer 132 is a base plate of a pump or a valve. The base plate may be a circular metal element that defines an inlet hole and an outlet hole. In some examples, the base plate may define an inlet hole, a channel through the base plate, and an outlet hole. In some examples, the conductive layer 132 is a passive valve layer. A passive valve layer may be a check valve with a helical slot and an opening. In some examples, the conductive layer 132 is a layer of a pressure sensor layer. In some examples, the conductive layer 132 of an actuator (e.g., an actuator diaphragm). The fluidic component 174 may include other layers such as one or more passive valve layers (e.g., check valve layers) defining one or more inlet valves or one or more outlet valves. The fluidic component 174 may include other layers such as an isolation layer that separates the actuator 130 and the base plate 432, and/or one or more adhesive layers (e.g., epoxy) that couple one or more layers together.

The conductive layer 132 includes a bonding rim 134 that is coupled to the fluidic manifold 108 or another component of the fluidic component 174. In some examples, the bonding rim 134 is a flap member. In some examples, the bonding rim 134 is a surface feature that extends around a perimeter of the conductive layer 132. In some examples, the bonding rim 134 may be coupled to the fluidic manifold 108 or another element of the fluidic component 174 based on a fusion weld (e.g., applying heat to metal components). The bonding rim 134 may reduce deformation to the fluidic component 174 and/or the fluidic manifold 108. The bonding rim 134 may be a portion (e.g., a perimeter or circumferential portion) of the conductive layer 132. In some examples, the conductive layer 132 may include a channel disposed around the perimeter of the conductive layer 132, where the bonding rim 134 is defined by the channel. In some examples, the bonding rim 134 includes a raised portion (or an extended portion) that extends from a surface or an edge of the conductive layer 132.

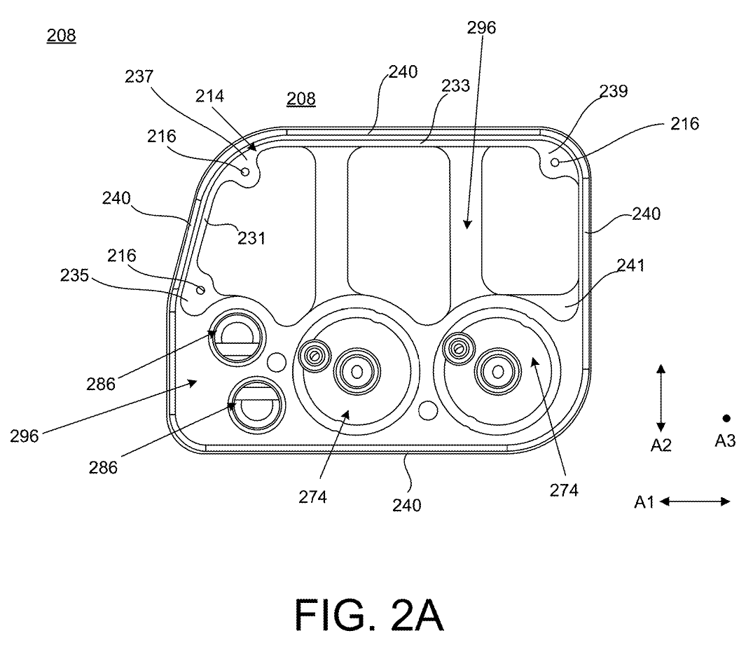

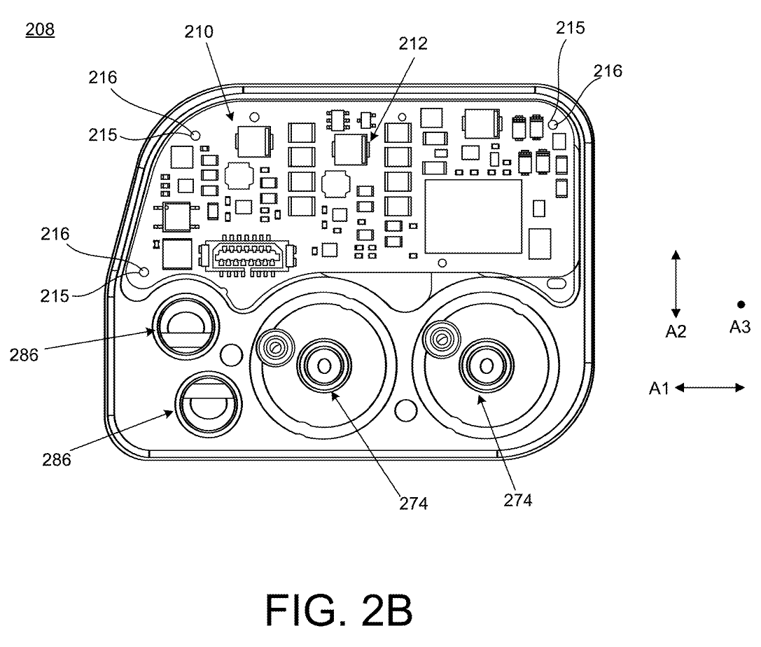

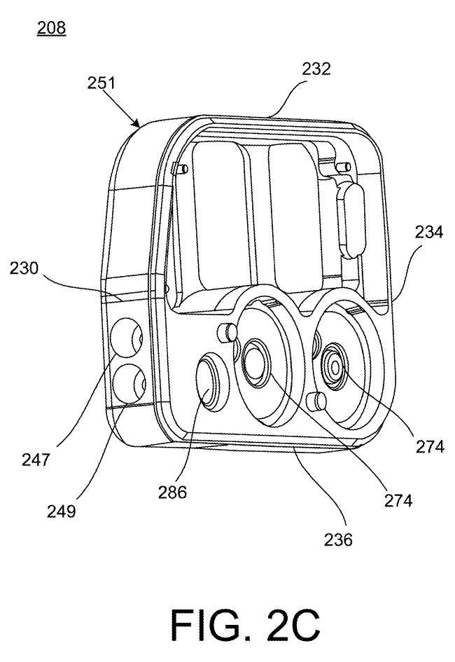

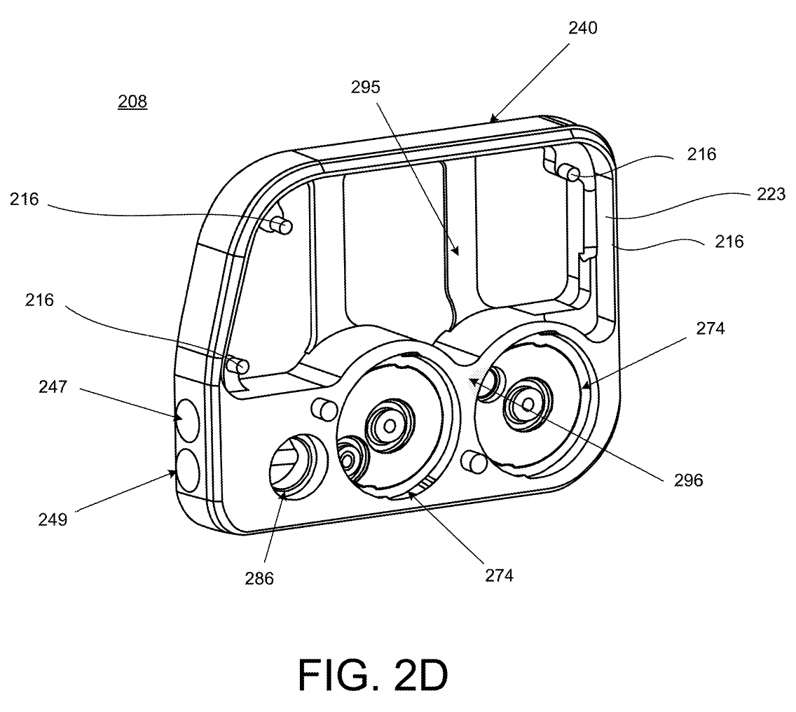

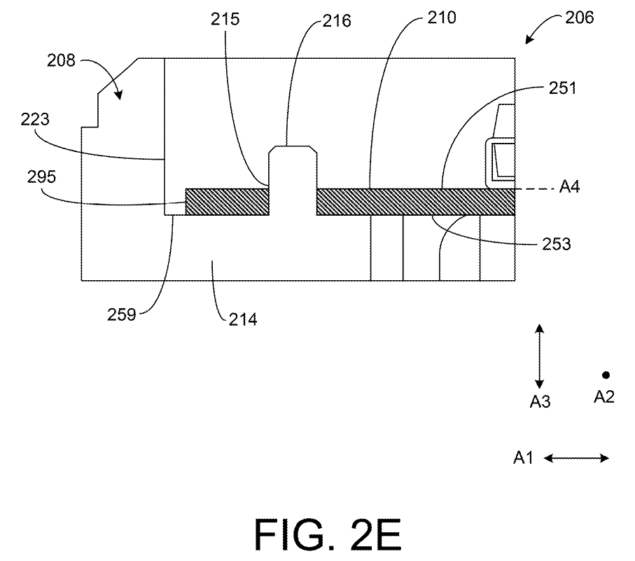

FIGS. 2A to 2E illustrates a fluidic manifold 208 according to an aspect. The fluidic manifold 208 may be an example of the fluidic manifold 108 of FIG. 1 and may include any of the details discussed with reference to FIG. 1. The fluidic manifold 208 includes a portion 296 configured to attach one or more pressure sensors 286 and one or more fluidic components 274 (e.g., pump(s), valves). The fluidic manifold 208 includes a portion 296 configured to attach a circuit substrate 210 with electronic components 212. In some examples, the circuit substrate 210 is a printed circuit board (PCB). The fluidic components 274 may include one or more pumps (e.g., electrically controlled pumps). The fluidic components 274 may include one or more valves. In some examples, the fluidic manifold 208 includes a conductive-based material (e.g., a metal-based material). In some examples, the fluidic manifold 208 is a titanium frame.

In some examples, the pressure sensors 286 include a first pressure sensor connected to a fluid reservoir (e.g., the fluid reservoir 102 of FIG. 1) to detect a pressure in the fluid reservoir, and a second pressure sensor connected to an inflatable member (e.g., the inflatable member 104 of FIG. 1) to detect a pressure in the inflatable member. In some examples, the bonding rim (e.g., the bonding rim 134 of FIG. 1) is formed on one or more of the layers of a pressure sensor 286.

The fluidic manifold 108 includes a frame 240 with an inside edge 223. The fluidic manifold 208 includes a shelf 214 that extends from the inside edge 223. The circuit substrate 210 contacts the shelf 214 (e.g., a surface 259 of the shelf 214). The circuit substrate 210 may sit on top of the shelf 214 such that the circuit substrate 210 is positioned within the frame 240. The circuit substrate 210 includes a first surface 251 and a second surface 253 that is opposite to the first surface 251. As shown in FIG. 2E, the first surface 251 of the circuit substrate 210 is disposed in the plane A4. The thickness of the circuit substrate 210 is defined as the distance between the first surface 251 and the second surface 253 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The second surface 253 of the circuit substrate 210 may contact at least a portion of the surface 259 of the shelf 214.

The fluidic manifold 208 includes one or more coupling members 216 that couples the circuit substrate 210 to the fluidic manifold 208 (e.g., to the shelf 214 of the fluidic manifold 208). In some examples, the coupling members 216 includes one or more male features (e.g., protrusions, posts, fasteners, rivets, etc.) and one or more female features (e.g., holes, slots, openings, etc.). The male feature may be defined on the fluidic manifold 208 (e.g., the shelf 214 of the fluidic manifold 208) and the female features may be circuit substrate 210. In some examples, the female feature may be defined on the fluidic manifold 208 (e.g., the shelf 214 of the fluidic manifold 208) and the male features may be defined on the circuit substrate 210.

In some examples, the coupling members 216 include protrusions (e.g., posts, cylindrical posts, grooved posts, extension members, etc.) that extend from the shelf 214 in the direction A3, and the protrusions extend through holes 215 on the circuit substrate 210. In some examples, the interaction between the protrusions and the holes 215 form a press-fit coupling mechanism. Although some examples use a press-fit coupling mechanism, the circuit substrate 210, and the fluidic manifold 208 may be coupled to each other based on other types of coupling mechanisms.

The shelf 214 may include one or more shelf portions (e.g., also referred to as shoulder portions) that extend from the inside edge 223 of the frame 240 in the directions A1 and A2. The shelf 214 may have a thickness that extends in the direction A3. In some examples, the shelf portions extend from the inside edge 223 in the direction A2 and/or the direction A3 at multiple different lengths. The shelf portions may include a corner portion 235, a corner portion 237, a corner portion 239, and a corner portion 241. The corner portion 235, the corner portion 237, and the corner portion 241 may define, include, or contact a coupling member 216. The shelf portions may include a connecting portion 231 that extends between the corner portion 235 and the corner portion 237. The shelf portions may include a connecting portion 233 that extends between the corner portion 237 and the corner portion 239.

The frame 240 may define a peripheral wall formed by a wall portion 230, a wall portion 232, a wall portion 234, and a wall portion 236. In some examples, the outer surface of the wall portion 230, the wall portion 232, the wall portion 234, and the wall portion 236 form a portion of the outer surface of the electronic pump device. The fluidic manifold 208 includes a first fluid port 247 and a second fluid port 249. The first fluid port 247 and the second fluid port 249 are defined on the wall portion 230. A first tube member (e.g., tube member 103 of FIG. 1) may be coupled to and extend from the first fluid port 247, and a second tube member (e.g., tube member 105 of FIG. 1) may be coupled to and extend from the second fluid port 249. In some examples, the outer surface of the wall portion 230, the wall portion 232, the wall portion 234, and the wall portion 236 form a portion of the outer surface of the housing (e.g., the housing 120 of FIG. 1).

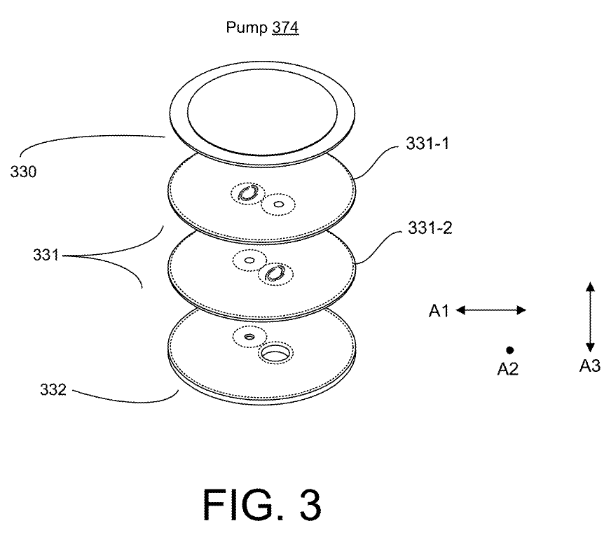

FIG. 3 illustrates an example of a pump 374. In some examples, the pump 374 is an electronically controlled pump. In some examples, the pump 374 includes a piezoelectric diaphragm pump. The pump 374 includes an actuator 330, one or more passive valve layers 331, and a base plate 332. In some examples, the actuator 330 includes an actuator layer (e.g., an actuator foil). In some examples, the actuator 330 includes a conductive-based material (e.g., a metal-based material).

The actuator 330 may receive an electrical signal, in response to the electrical signal, the actuator 330 may deform (e.g., bend, change its shape, etc.). The actuator 330 may be one or more multiple components that are coupled together. In some examples, the actuator 330 includes one or more disc-shaped elements. In some examples, the actuator 330 includes an activation element (e.g., a piezo element with one or more electrodes, a piezo disc actuator, etc.) and an actuator diaphragm coupled to the activation element. The actuator diaphragm may be a flexible membrane. In some examples, the actuator diaphragm may be a metal-based flexible membrane. In some examples, the actuator 330 includes a piezo disc actuator, an isolation layer, an adhesive material, and an actuator diaphragm. An electrical signal applied to the activation element may cause the activation element to deform, thereby deforming the actuator diaphragm (e.g., bend, change shape). For example, when an electrical signal is applied to the electrodes of the activation element, the activation element undergoes a physical deformation, and this deformation is transmitted to the actuator diaphragm, causing it to move. The movement of the actuator diaphragm controls the flow of fluid through the pump 374.

In some examples, the passive valve layers 331 include disc layers or circular layers. In some examples, a passive valve layer 331 includes a foil layer. A passive valve layer 331 includes a fluid control element at a location on the passive valve layer 331. The fluid control element may be a feature on the passive valve layer 331 that causes fluid to flow through the passive valve layer 331 in one direction (but not the other direction). In some examples, the fluid control element is a check valve. In some examples, the fluid control element includes an input check valve. In some examples, the fluid control element includes an output check valve. The fluid control element may include one or more slots. In some examples, the fluid control element includes a helical slot. In some examples, the fluid control element is aligned with one of the holes of the base plate 332.

In some examples, the base plate 332 includes a disc layer or a circular layer. The base plate 332 includes a cylindrical disc. In some examples, the base plate 332 includes a conductive material (e.g., a metal-based material). In some examples, a portion of the base plate 332 is coupled to the fluidic manifold. The base plate 332 may include a first hole (e.g., a through hole) and a second hole (e.g., a through hole). The base plate 332 includes a first surface and a second surface disposed opposed to the first surface. The distance between the first surface and the second surface may define a thickness of the base plate in a direction A3.

In some examples, the passive valve layers 331 include a passive valve layer 331-1 and a passive valve layer 331-2. In some examples, the passive valve layers 331 may be disposed between the actuator 330 and the base plate 332. In some examples, one or more passive valve layers 331 are coupled to the bottom of the base plate 332 (e.g., the bottom of the base plate 332 is the surface of the base plate 332 that faces the recessed surface of the fluidic manifold). In some examples, the passive valve layer 331-2 is coupled to the base plate 332, and the passive valve layer 331-2 is coupled to the passive valve layer 331-1. In some examples, the actuator 330 is coupled to the passive valve layer 331-1.

In some examples, the conductive layer 132 of FIG. 1 is an example of the base plate 332. In some examples, the conductive layer 132 of FIG. 1 is an example of the passive valve layer 331-1. In some examples, the conductive layer 132 of FIG. 1 is an example of the passive valve layer 331-2. In some examples, the conductive layer 132 of FIG. 1 is an example of the actuator 330.

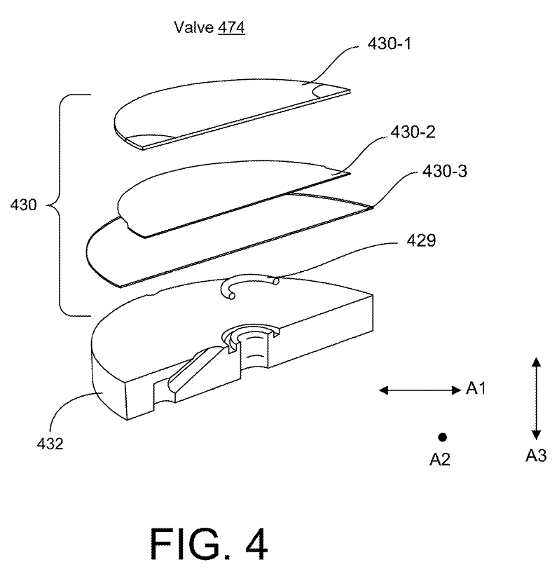

FIG. 4 illustrates an example of a valve 474. In some examples, the valve 474 is an active valve. In some examples, the valve 474 is an electronically controlled valve. In some examples, the valve 474 includes a passive valve. The valve 474 includes an actuator 430 and a base plate 432. In some examples, the actuator 430 includes an activation element 430-1, an actuator layer 430-2 (e.g., an adhesive layer, isolation layer, or other types of intermediate layer), an actuator diaphragm 430-3. The actuator 430 (e.g., the activation element 430-1, the actuator diaphragm 430-3) may include any of the details described with reference to FIGS. 1 and 3. In some examples, the valve 474 includes a ring member 429 (e.g., an O-ring) that is inserted into a slot of the base plate 432 (e.g., the slot may be formed on the intersection of the base plate 432 and the actuator 430). In some examples, a portion of the base plate 432 is coupled to the fluidic manifold. In some examples, the base plate 432 is a component that is integral to the fluidic manifold. In some examples, the conductive layer 132 of FIG. 1 is an example of the base plate 432. In some examples, the conductive layer 132 of FIG. 1 is an example of the actuator 430 (e.g., the actuator diaphragm 430-3).

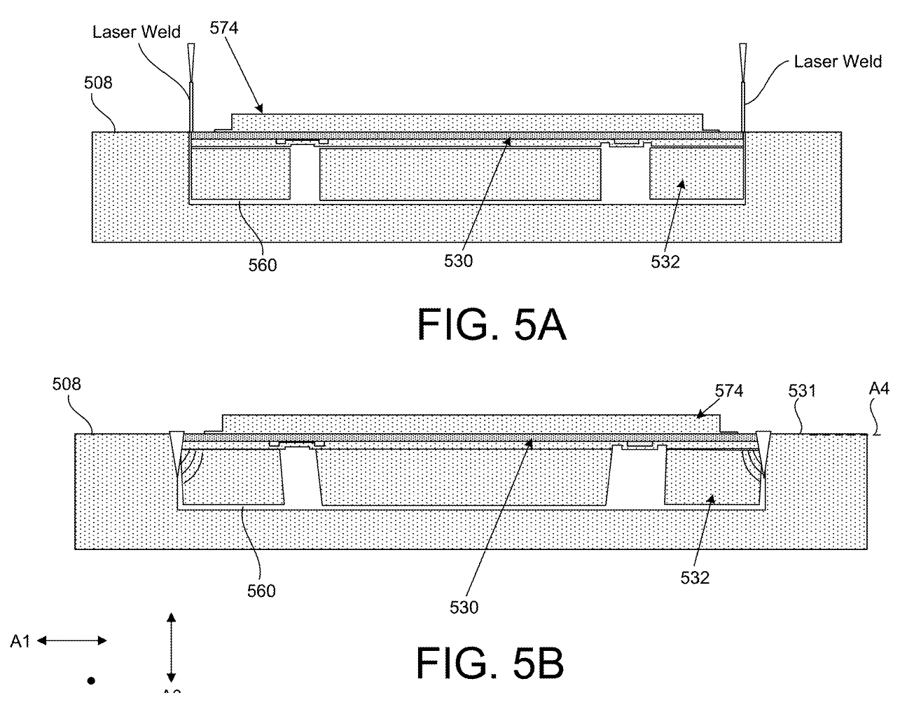

FIG. 5A illustrates an example of a fluidic component 574 disposed within a recess 560 of a fluidic manifold 508. In some examples, the recess 560 may be a three-dimensional cavity or depression formed in the fluidic manifold 508 configured to receive and accommodate at least a portion of the fluidic component 574. In some examples, the recess 560 may have a width defined by the distance between the opposing walls of the recess 560, measured perpendicular to the recess's depth. In some examples, the recess 560 may have a depth defined by the distance between the recess's base and the plane of the fluidic manifold's surface, measured perpendicular to the surface.

In some examples, the fluidic component 574 includes a pump such as the pump 374 of FIG. 3. In some examples, the fluidic component 574 includes a valve such as the valve 474 of FIG. 4. In some examples, the fluidic component 574 may be a pressure sensor. The fluidic component 574 includes a conductive layer (e.g., the conductive layer 132 of FIG. 1) such as an actuator 530 or a base plate 532 (or a passive valve layer). In some examples, the base plate 532 is the base plate 332b of FIG. 3. In some examples, the base plate 532 is the base plate 432 of FIG. 4. FIG. 5B illustrates the fluidic component 574 being coupled to the fluidic manifold 508 based on a fusion weld. A fusion weld is coupling mechanism that joins materials by melting them together.

The fluidic manifold 508 includes a surface 531. The recess 560 may be an opening that extends in a direction A3 from the surface 531. The surface 531 is disposed in a plane A4. The direction A3 is orthogonal to the plane A4. The direction A1 is perpendicular to the direction A3 and perpendicular to the direction A2. The direction A2 is perpendicular to the direction A1 and perpendicular to the direction A3.

Referring to FIG. 5A, the fluidic component 574 is disposed in the recess 560 of the fluidic manifold 508. Referring to FIG. 5B, heat is applied to the edge portions of the fluidic component 574. In some examples, the application of heat may cause deformation to one or more components of the fluidic component 574. However, the bonding rim (e.g., the bonding rim 134 of FIG. 1) of one or more conductive layers (e.g., a base plate or another layer) may be used to reduce the potential deformation of the fluidic component 574 caused by the application of heat to bond the fluidic component 574 to the fluidic manifold 508.

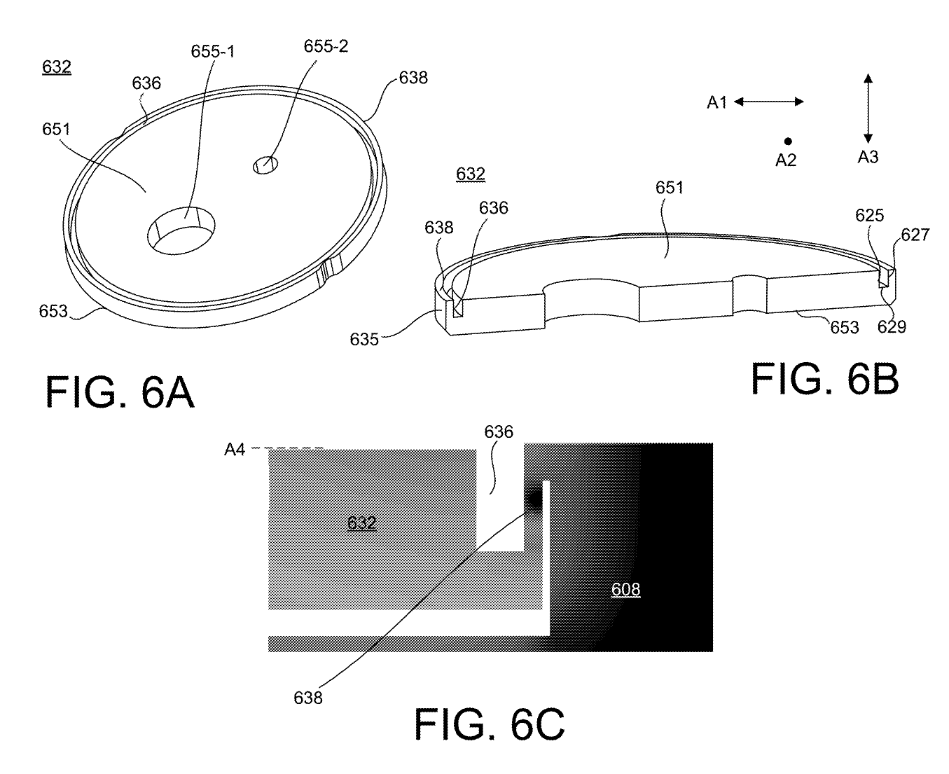

FIGS. 6A to 6C illustrate a base plate 632 according to an aspect. The base plate 632 may be an example of the base plate 332b of FIG. 3 or the base plate 432 of FIG. 4. Although a base plate 632 is illustrated in FIGS. 6A to 6C, a bonding rim 638 may be formed on any type of conductive layer. The base plate 632 may be a circular disc. In some examples, the base plate 632 may be a cylinder portion. In some examples, the base plate 632 includes a hole 655-1 (e.g., a through-hole). In some examples, the base plate 632 includes a hole 655-2 (e.g., a through-hole). The hole 655-1 and the hole 655-2 may be an inlet port and an outlet port, respectively (or vice versa). In some examples, the size of the hole 655-2 is smaller than the size of the hole 655-1. The base plate 632 includes a surface 651 and a surface 653 disposed opposite to the surface 651. The base plate 632 includes an edge 635 that extends between the surface 651 and the surface 653 in the direction A3. The edge 635 may define the outermost edge around the disc. In some examples, the distance between the surface 651 and the surface 653 defines a thickness of the base plate 632 in a direction A3. In some examples, the hole 655-1 and the hole 655-2 extends between (and through) the surface 651 and the surface 653.

The surface 651 is aligned with a plane A4. The direction A3 is orthogonal to the plane A4. The direction A1 is perpendicular to the direction A3 and perpendicular to the direction A2. The direction A2 is perpendicular to the direction A1 and perpendicular to the direction A3.

The base plate 632 includes a bonding rim 638. In some examples, the bonding rim 638 is referred to as a lip or a circumferential lip. In some examples, the bonding rim 638 can be referred to as a flap. The bonding rim 638 is coupled to a fluidic manifold 608. In some examples, the bonding rim 638 is coupled to the fluidic manifold 608 based on a fusion weld. For example, heat may be applied to the bonding rim 638, which bonds the bonding rim 638 to the fluidic manifold 608.

The base plate 632 includes a channel 636 disposed around a perimeter portion of the base plate 632. The channel 636 may be a groove. The channel 636 may be a depression. In some examples, the channel 636 includes a three-dimensional groove or depression formed in the base plate 632. The channel 636 may extend along a portion or the entirety of the base plate's perimeter. In some examples, the width of the channel 636 may be the distance between the opposing walls of the channel 636, measured perpendicular to the channel's length. In some examples, the depth of the channel 636 may be the distance between the channel's base and the plane of the base plate's first surface, measured perpendicular to the first surface. In some examples, the length of the channel 636 may be the extent of the channel along the base plate's perimeter.

The channel 636 may be considered a slot, an opening, or a cutout portion that extends around the perimeter portion of the base plate 632. The channel 636 may be defined by an edge 625 that extends along the direction A3, an edge 627 that extends along the direction A3, and edge 629 that extends in the direction A1. In some examples, the channel 636 extends from the surface 651 and into the thickness of the base plate 632 in the direction A2. The bonding rim 638 is defined by the channel 636 and at least a portion of the edge 635.

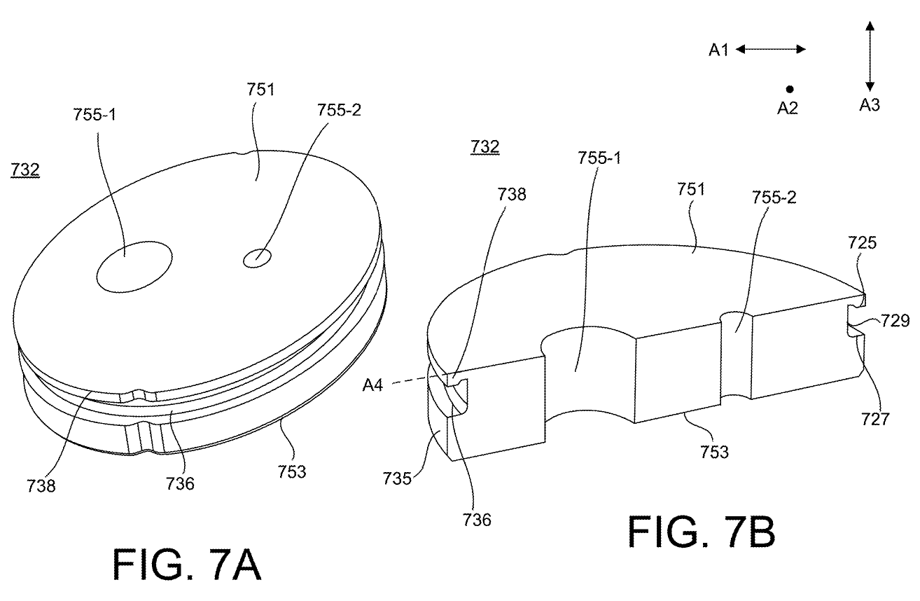

FIGS. 7A and 7B illustrate a base plate 732 according to an aspect. The base plate 732 may be an example of the base plate 332b of FIG. 3 or the base plate 432 of FIG. 4. Although a base plate 732 is illustrated in FIGS. 7A and 7B, a bonding rim 738 may be formed on any type of conductive layer. The base plate 732 may be a circular disc. In some examples, the base plate 732 may be a cylinder portion. In some examples, the base plate 732 includes a hole 755-1 (e.g., a through-hole). In some examples, the base plate 732 includes a hole 755-2 (e.g., a through-hole). The hole 755-1 and the hole 755-2 may be an inlet port and an outlet port, respectively (or vice versa). In some examples, the size of the hole 755-2 is smaller than the size of the hole 755-1. The base plate 732 includes a surface 751 and a surface 753 disposed opposite to the surface 751 in the direction A3. The base plate 732 includes an edge 735 that extends between the surface 751 and the surface 733 in the direction A3. In some examples, the distance between the surface 751 and the surface 753 defines a thickness of the base plate 732 in the direction A3. In some examples, the hole 755-1 and the hole 755-2 extends between (and through) the surface 751 and the surface 753.

The surface 751 is aligned with a plane A4. The direction A3 is orthogonal to the plane A4. The direction A1 is perpendicular to the direction A3 and perpendicular to the direction A2. The direction A2 is perpendicular to the direction A1 and perpendicular to the direction A3.

The base plate 732 includes a bonding rim 738. In some examples, the bonding rim 738 is referred to as a lip or a circumferential lip. In some examples, the bonding rim 638 can be referred to as a flap. The bonding rim 738 is coupled to a fluidic manifold. In some examples, the bonding rim 738 is coupled to the fluidic manifold based on a fusion weld. For example, heat may be applied to the bonding rim 738, which bonds the bonding rim 738 to the fluidic manifold.

The base plate 732 includes a channel 736 disposed around a perimeter portion of the base plate 732. The channel 736 may be considered a slot, an opening, or a cutout portion that extends around the perimeter portion of the base plate 732. In some examples, the channel 736 may be a three-dimensional groove or depression formed in the side or edge of the base plate 732, extending along a portion or the entirety of the base plate's outer edge. In some examples, the channel 736 does not extend into the first surface 751 of the base plate 732. In some examples, the channel 736 has a width defined by the distance between the opposing walls of the channel, measured perpendicular to the channel's length. In some examples, the channel 736 has a depth defined by the distance between the channel's base and the outer edge of the base plate, measured perpendicular to the edge. In some examples, the channel 736 has a length defined by the extent of the channel 736 along the base plate's outer edge.

In some examples, the channel 736 extends from the edge 735 and into the side of the base plate 732 in the direction A3. The channel 736 may be defined by an edge 725, an edge 727, and an edge 729. The edge 725 (or a portion thereof) and the edge 727 (or a portion thereof) may be parallel to each other and may extend in the direction A1. The edge 729 may be perpendicular to the edge 725 (or a portion thereof) and to the edge 737 (or a portion thereof). The edge 729 may extend in the direction A3.

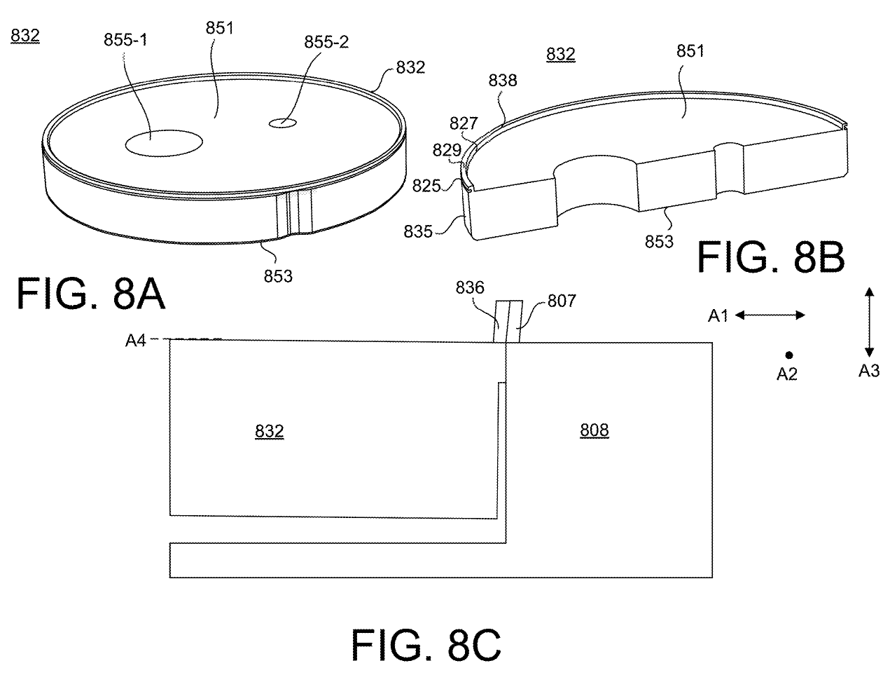

FIGS. 8A to 8C illustrate a base plate 832 according to an aspect. The base plate 832 may be an example of the base plate 332b of FIG. 3 or the base plate 432 of FIG. 4. Although a base plate 832 is illustrated in FIGS. 8A to 8C, a bonding rim 838 may be formed on any type of conductive layer. The base plate 832 may be a circular disc. In some examples, the base plate 832 may be a cylinder portion. In some examples, the base plate 832 includes a hole 855-1 (e.g., a through-hole). In some examples, the base plate 832 includes a hole 855-2 (e.g., a through-hole). In some examples, the size of the hole 855-2 is smaller than the size of the hole 855-1. The hole 855-1 and the hole 855-2 may be an inlet port and an outlet port, respectively (or vice versa). The base plate 832 includes a surface 851 and a surface 853 disposed opposite to the surface 851. The base plate 832 includes an edge 835 that extends between the surface 851 and the surface 833 in the direction A3. The edge 835 may define the outermost edge around the disc. In some examples, the distance between the surface 851 and the surface 853 defines a thickness of the base plate 832 in a direction A3. In some examples, the hole 855-1 and the hole 855-2 extends between (and through) the surface 851 and the surface 853.

The surface 851 is aligned with a plane A4. The direction A3 is orthogonal to the plane A4. The direction A1 is perpendicular to the direction A3 and perpendicular to the direction A2. The direction A2 is perpendicular to the direction A1 and perpendicular to the direction A3.

The base plate 832 includes a bonding rim 838. In some examples, the bonding rim 838 is referred to as a lip or a circumferential lip. In some examples, the bonding rim 638 can be referred to as a flap. In some examples, the bonding rim 838 includes a raised portion. In some examples, the bonding rim 838 includes an extension to the edge 835. The bonding rim 838 is coupled to a fluidic manifold 808. In some examples, the bonding rim 838 is coupled to the fluidic manifold 808 based on a fusion weld. For example, heat may be applied to the bonding rim 838, which bonds the bonding rim 838 to the fluidic manifold 808. The bonding rim 838 may be a raised portion that extends around the perimeter of the base plate 832. In some examples, the bonding rim 838 may be defined by an edge 825, an edge 827, and an edge 829. The edge 825 (or a portion thereof) and the edge 827 (or a portion thereof) may be parallel to each other and may extend in the direction A1. The edge 829 may be perpendicular to the edge 825 (or a portion thereof) and to the edge 827 (or a portion thereof). The edge 829 may extend in the direction A3.

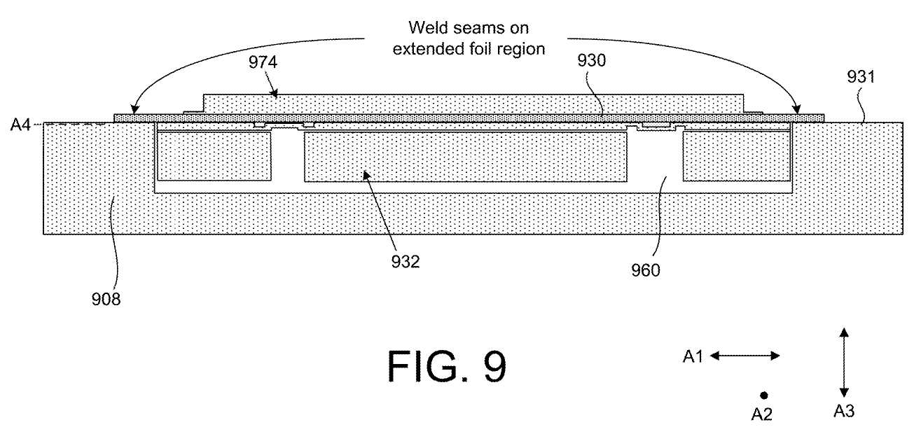

FIG. 9 illustrates an example of a fluidic component 974 disposed within a recess 960 of a fluidic manifold 908. In some examples, the fluidic component 974 includes a pump such as the pump 374 of FIG. 3. In some examples, the fluidic component 974 includes a valve such as the valve 474 of FIG. 4. In some examples, the fluidic component 974 is a pressure sensor. The fluidic component 974 includes an actuator 930 and a base plate 932. In some examples, the actuator 930 includes a size (e.g., a diameter) that extends in a direction A1 that is greater than a size (e.g., a diameter) of the base plate 932. In some examples, the base plate 932 is the base plate 332b of FIG. 3. In some examples, the base plate 932 is the base plate 432 of FIG. 4. The fluidic component 974 is coupled to the fluidic manifold 908 based on a fusion weld.

In some examples, the actuator 930 is fusion welded to a surface 931 of the fluidic manifold 908. The recess 960 may be an opening that extends in a direction A3 from the surface 931. The surface 931 is disposed in a plane A4. The direction A3 is orthogonal to the plane A4. The direction A1 is perpendicular to the direction A3 and perpendicular to the direction A2. The direction A2 is perpendicular to the direction A1 and perpendicular to the direction A3. The base plate 932 is disposed in the recess 960 and a portion of the actuator 930 contacts the surface 931 of the fluidic manifold 908. Heat is applied to the edge portion of the actuator 930 but may minimize deformation to the base plate 932.

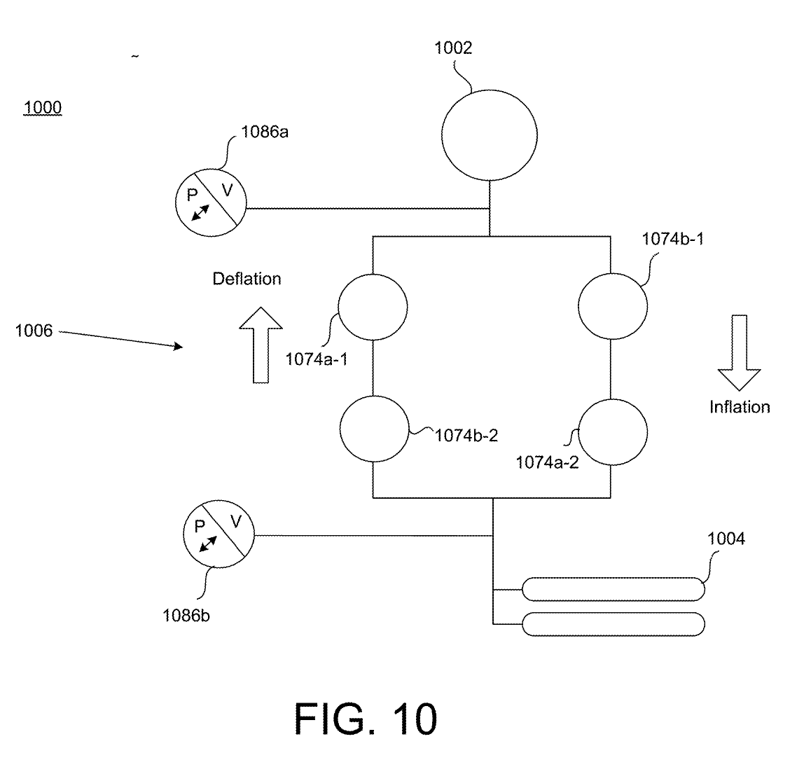

FIG. 10 illustrates an inflatable medical device 1000 according to an aspect. In some examples, the implantable medical device 1000 is an inflatable penile prosthesis. The implantable medical device 1000 includes a fluid reservoir 1002, an inflatable member 1004, and an electronic pump device 1006 configured to transfer fluid between the fluid reservoir 1002 and the inflatable member 1004 to inflate or deflate the inflatable member 1004. In some examples, the inflatable member 1004 is a penile inflation member (e.g., one or more inflatable cylinders) that may be implanted into the corpus cavernosum of the user.

The electronic pump device 1006 includes a pressure sensor 1086a connected to the fluid reservoir 1002 and configured to monitor a pressure of the fluid reservoir 1002, and a pressure sensor 1086b connected to the inflatable member 1004 and configured to monitor a pressure of the inflatable member 1004. The electronic pump device 1006 includes a valve 1074a-1 and a pump 1074b-2. The valve 1074a-1 and the pump 1074b-2 may be used to deflate the inflatable member 1004. The electronic pump device 1006 includes a valve 1074a-2 and a pump 1074b-1. The valve 1074a-2 and the pump 1074b-1 may be used to inflate the inflatable member 1004. The valve 1074a-1 may include a base plate according to any of the examples discussed herein. The pump 1074b-2 may include a base plate according to any of the examples discussed herein. The valve 1074a-2 may include a base plate according to any of the examples discussed herein. The pump 1074b-1 may include a base plate according to any of the examples discussed herein.

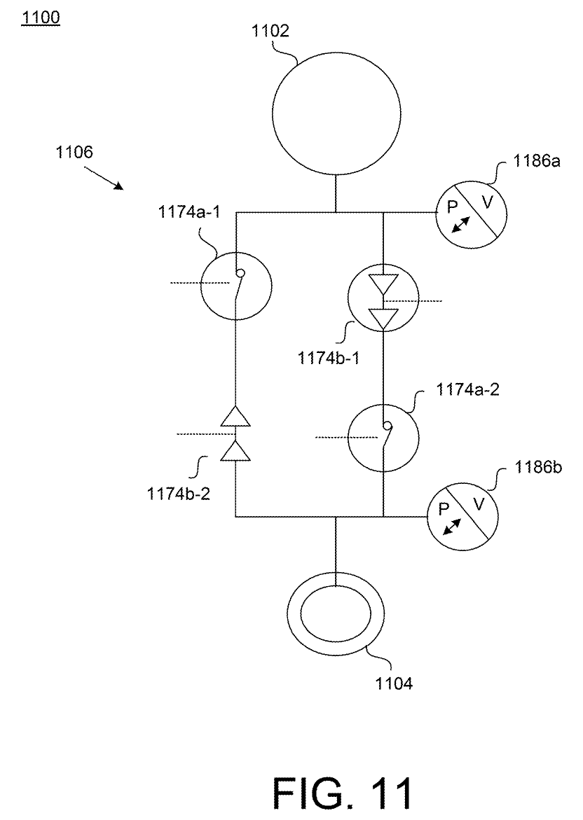

FIG. 11 illustrates an inflatable medical device 1100 according to an aspect. In some examples, the implantable medical device 1100 is an artificial urinary sphincter device. The implantable medical device 1100 includes a fluid reservoir 1102, an inflatable member 1104, and an electronic pump device 1106 configured to transfer fluid between the fluid reservoir 1102 and the inflatable member 1104 to inflate or deflate the inflatable member 1104. In some examples, the inflatable member 1104 is an inflatable cuff.

The electronic pump device 1106 includes a pressure sensor 1186a connected to the fluid reservoir 1102 and configured to monitor a pressure of the fluid reservoir 1102, and a pressure sensor 1186b connected to the inflatable member 1104 and configured to monitor a pressure of the inflatable member 1104. The electronic pump device 1106 includes a valve 1174a-1 and a pump 1174b-2. The valve 1174a-1 and the pump 1174b-2 may be used to deflate the inflatable member 1104. The electronic pump device 1106 includes a valve 1174b-1 and a pump 1174b-1. The valve 1174a-2 and the pump 1174b-1 may be used to inflate the inflatable member 1104. The valve 1174a-1 may include a base plate according to any of the examples discussed herein. The pump 1174b-2 may include a base plate according to any of the examples discussed herein. The valve 1174a-2 may include a base plate according to any of the examples discussed herein. The pump 1174b-1 may include a base plate according to any of the examples discussed herein.

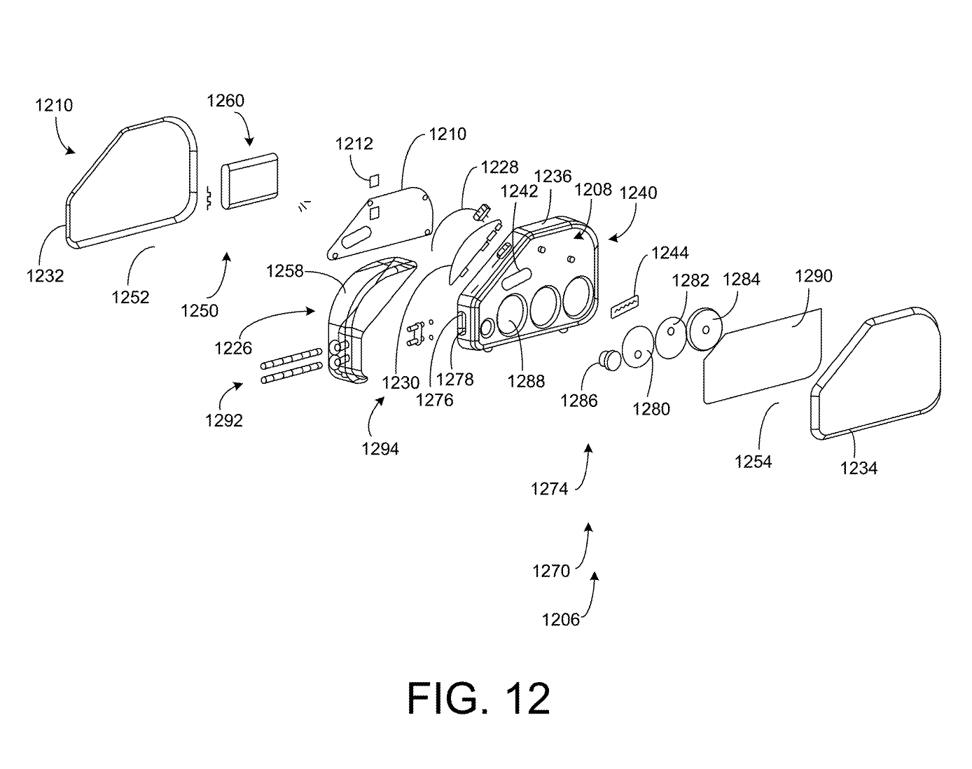

FIG. 12 illustrates an example of an exploded view of an electronic pump device 1206 according to an aspect. The housing 1220 includes a first sidewall 1232, a second sidewall 1234, a peripheral wall 1236, and a frame 1240. The first sidewall 1232, second sidewall 1234, and the peripheral wall 1236 are hermetically sealed together to form an internal compartment 1250 within the housing 1220.

The frame 1240 is disposed within the internal compartment 1250 to form a first partition 1252 and a second partition 1254 in such a manner that the first partition 1252 is hermetically sealed from the second partition 1254. The frame 1240 can be integrally formed with the peripheral wall 1236, the first sidewall 1232, and/or the second sidewall 1234. In some examples, the frame 1240 is welded to the peripheral wall 1236 or welded to the first sidewall 1232, and/or the second sidewall 1234. The first sidewall 1232, the peripheral wall 1236, and the frame 1240 may form the first partition 1252. The second sidewall 1234, the peripheral wall 1236, and the frame 1240 may form the second partition 1254, which is opposite the frame 1240 from the first partition 1252.

The electronic pump device 1206 can include a header 1226 attached to the housing 1220 to form an internal region 1258 between an inner surface of the header 1226 and an outer surface of the housing 1220 that includes power and communication interface structures such as a secondary coil 1228 and the antenna 1230 external to the hermetically sealed housing 1220. The header 1226 is configured from a dielectric or insulative material, such as a radome, to allow the transmission of power and communication signals between the antenna 1230 and a handset programmer or charger, and between the secondary coil 1228 and the charger. For example, the header 1226 may include an over-molded polymer affixed to the housing 1220 and including the secondary coil 1228 and the antenna 1230 within the internal region 1258. The secondary coil 1228 and antenna 1230 are constructed from a biocompatible material. In some examples, the secondary coil 1228 and antenna 1230 can be formed as a coil from a stamped titanium core clad with gold or silver. In some examples, the secondary coil 1228 and antenna 1230 can be formed from a gold wire.

The electronic pump device 1206 includes an energy storage system, such as a battery (e.g., a rechargeable power source) (e.g., a rechargeable battery), and electronic components 1212 within the first partition 1252. The electronic components 1212 can be disposed on a circuit substrate 1210, such as a plurality of circuit boards, within the first partition 1252. The battery 1260 can assume various forms appropriate to provide power for generating desired electrical signals and to store power provided from the electronic components 1212. For example, the battery 1260 can incorporate lithium-ion (Li+) chemistry, e.g., a lithium-ion battery to operate the electronic components 1212. In some examples, the electronic components 1212 can be implemented by various components including resistors, capacitors, transistors, and integrated circuits disposed on the circuit substrate 1210. The secondary coil 1228 and antenna 1230 are electrically coupled to the electronic components 1212 within the first partition 1252, such as via a hermetic feedthrough component.

The electronic components 1212 can include a recharge system, a communication system, and a controller. The recharge system includes hardware configured to interface with the secondary coil 1228 to receive power signals, and to provide the power signals in a form suitable to recharge the battery 1260 and can include circuitry to reduce the likelihood of overcharging the battery 1260. The communication system includes hardware configured to interface with the antenna 1230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., external device 101) (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna 1230 for transmission. In some examples, the communication system can be configured to receive and transmit radio frequency signals via the antenna 1230. The controller can include a microcontroller to operate the recharge system and to receive and operate in response to communication signals or generate communication signals from the communication system.

The electronic pump device 1206 also includes a fluidic circuit 1270 within the second partition 1254 and opposite the frame 1240 from the battery 1260 and electronic components 1212. In some examples, the frame 1240 can include an opening 1242 that includes a hermetic interface 1244, such as a feedthrough hermetically affixed to the frame 1240. The electronic components 1212 are operably coupled to the fluidic circuit 1270 across the frame 1240 via the hermetic interface 1244. For example, the controller of the electronic components 1212, powered by the battery 1260, can cause the operation of the fluidic circuit 1270 such as to control and monitor the fluidic circuit 1270.

The fluidic circuit 1270 includes a fluidic manifold 1208 and fluidic components 1274 operably coupled to the fluidic manifold 1208. In some examples, the fluidic manifold 1208 is a structure integrated into the frame 1240 such that the fluidic manifold 1208 and the frame 1240 together form the hermetic barrier between the first partition 1252 and the second partition 1254 of the internal compartment 1250. For instance, the battery 1260, the circuit substrate 1210, or electronic components 1212 can be coupled to a first major surface of the fluidic manifold 1208 in the first partition 1252, and the fluidic components 1274 are operably coupled to a second, and opposite major surface of the fluidic manifold 1208 in the second partition 1254.

The fluidic circuit 1270 provides for the transfer of the fluid between the fluid reservoir (e.g., the fluid reservoir 102 of FIG. 1) and the inflatable member (e.g., the inflatable member 104 of FIG. 1). The fluidic manifold 1208, which can be a hermetic manifold, segments and contains the fluid from the internal compartment 1250 to reduce the chance of fluid exchange and directs the fluid from a first port 1276 to a second port 1278 via internal fluid passageways or channels.

The fluidic components 1274 include a plurality of fluid pumps, such as pumps 1280, 1282, a valve 1284 mounted into the fluidic manifold 1208 in fluidic communication with a manifold passageway to transfer fluid from the first port 1276 to the second port 1278. The pumps and the valve(s) are in fluid communication with a single fluid passageway between ports 1276, 1278. The fluidic components 1274 also includes one or more pressure sensors 1286 operably coupled to the fluidic manifold 1208 and in fluidic communication with the passageway to detect a pressure of the fluid within the fluidic manifold 1208.

The fluidic components 1274 are included in a planar configuration on the fluidic manifold 1208 in which the pumps 1280, 1282, valve 1284, and pressure sensor 1286 are mounted into the fluidic manifold 1208 on a plane for slim profile within the second partition 1254. The fluidic manifold 1208 can include chambers 1288 formed into the second major surface in which the chambers are fluidically coupled to the single passageway within the fluidic manifold 1208. The chambers are configured to receive the pumps 1280, 1282, and valve 1284 and one or more pressure sensors 1286. In some examples, the fluidic manifold 1208 can receive a piezoelectric pump. The fluidic manifold 1208 can receive a component cover 1290 over the fluidic components 1274, which can be hermetically sealed to the second major surface.

In some examples, the electronic pump device 1206 may include kink resistant tubing 1292 that can extend through the header 1226 and attached to the ports 1276, 1278 via components such as a barb 1294 and O-rings. The kink resistant tubing 1292 can be attached to the tube members 103, 105 of FIG. 1 to fluidically couple the electronic pump device 1206 to the fluid reservoir (e.g., the fluid reservoir 102 of FIG. 1) and the inflatable member (e.g., the inflatable member 104 of FIG. 1).

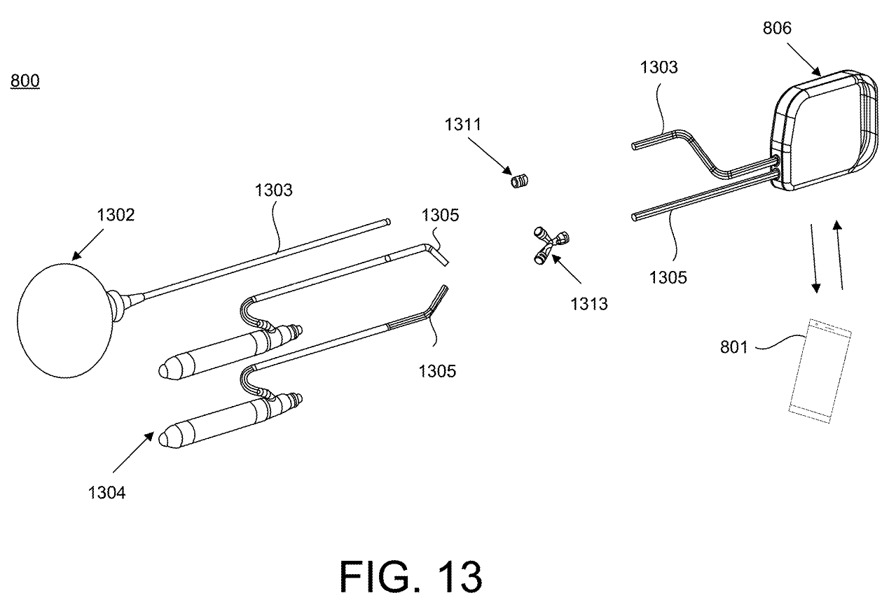

FIG. 13 illustrates a perspective of an inflatable penile prosthesis 1300 according to an aspect. The inflatable penile prosthesis 1300 may be an example of any of the medical devices discussed herein (e.g., including implantable medical device 100), and, therefore, may include any of the details discussed with reference to the previous figures.

The inflatable penile prosthesis 1300 includes an inflatable member 1304, a fluid reservoir 1302, and an electronic pump device 1306. The inflatable member 1304 includes a pair of inflatable cylinders. The electronic pump device 1306 may be an example of any of the pump devices discussed with reference to the previous figures and may include any of the details discussed herein. The electronic pump device 1306 includes fluidic components such as pumps, valves, and/or sensing devices positioned in fluid passageways. The electronic pump device 1306 includes components such as, for example, one or more fluid control devices, one or more pressure sensors, and other such components. The electronic pump device 1306 includes an electronic control system configured to provide for the transfer of fluid between a fluid reservoir 1302 and an inflatable member 1304 via the fluidic components.

The electronic pump device 1306 may include one or more integrated circuits. In some examples, the integrated circuits are included in a printed circuit board that is included in a housing of the electronic pump device 1306. Fluidic components and the electronic components of the electronic pump device 1306 are included in a housing. In some examples, fluidic components and electronic components in the housing define a manifold (e.g., an electronically controlled fluidic manifold) that provides for the electronic control of the flow of fluid between the fluid reservoir 1302 and the inflatable member 1304. In some examples, the electronic pump device 1306 can communicate with an external device 1301, via respective communication modules. For example, an application stored in a memory and executed by a processor of the external device 1301 may allow the user and/or a physician to operate, view, monitor and alter operation of the inflatable penile prosthesis 1300.

The inflatable penile prosthesis 1300 includes one or more first tube members 1303 that connect a first fluid port of the electronic pump device 1306 with the fluid reservoir 1302. One or more second tube members 1305 connect a second fluid port of the electronic pump device 1306 with the inflatable member 1304 in the form of the inflatable cylinders. In some examples, the inflatable penile prosthesis 1300 includes a connector 1311 that is used to connect two tube members 1303 together, and a connector 1313 that is used to connect two tube members 1305 together.

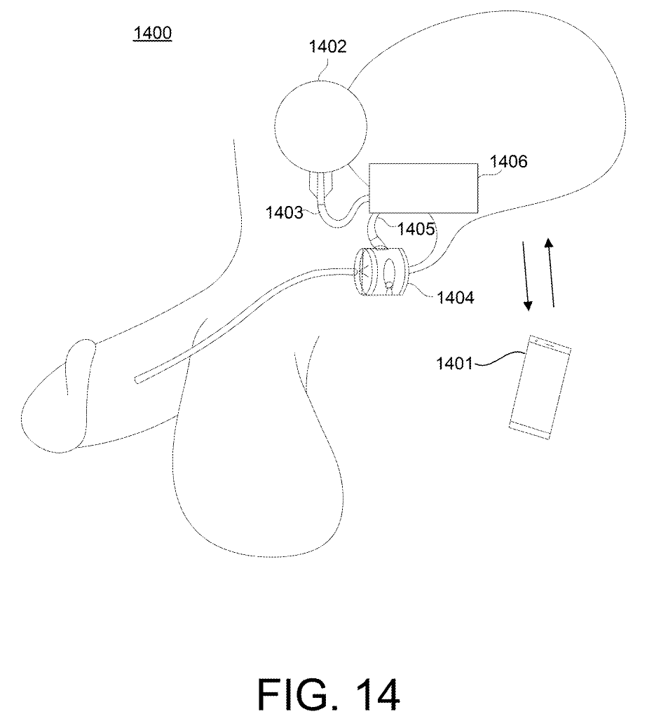

FIG. 14 illustrates a urinary control device 1400 having an electronic pump device 1406 according to an aspect. The urinary control device 1400 may be an example of the implantable medical device 100. In some examples, the urinary control device 1400 is an artificial urinary sphincter device. The electronic pump device 1406 may include any of the features of the pump devices discussed herein. The urinary control device 1400 includes an electronic pump device 1406, a fluid reservoir 1402, and a cuff 1404 (e.g., an inflatable cuff).

The fluid reservoir 1402 may be a pressure-regulating inflation balloon or element. The fluid reservoir 1402 is in operative fluid communication with the cuff 1404 via one or more tube members 1403, 1405. The fluid reservoir 1402 is constructed of polymer material that is capable of elastic deformation to reduce fluid volume within the fluid reservoir 1402 and push fluid out of the fluid reservoir 1402 and into the cuff 1404. However, the material of the fluid reservoir 1402 can be biased or include a shape memory construct adapted to generally maintain the fluid reservoir 1402 in its expanded state with a relatively constant fluid volume and pressure. In some examples, this constant level of pressure exerted from the fluid reservoir 1402 to the cuff 1404 will keep the cuff 1404 at a desired inflated state when open fluid communication is provided between the fluid reservoir 1402 and the cuff 1404. In some examples, the fluid reservoir 1402 is implanted into the abdominal space.

A user may use an external device 1401 to control the urinary control device 1400. In some examples, the user may use the external device 1401 to inflate or deflate the cuff 1404. For example, in response to the user activating an inflation cycle using the external device 1401, the external device 1401 may transmit a wireless signal to the electronic pump device 1406 to initiate the inflation cycle to transfer fluid from the fluid reservoir 1402 to the cuff 1404 (e.g., by opening an active valve where the pressure in the fluid reservoir 1402 causes the fluid to move through the active valve to the cuff 1404). In some examples, in response to the user activating a deflation cycle using the external device 1401, the external device 1401 may transmit a wireless signal to the pump device 1406 to initiate the deflation cycle to transfer fluid from the cuff 1404 to the fluid reservoir 1402.

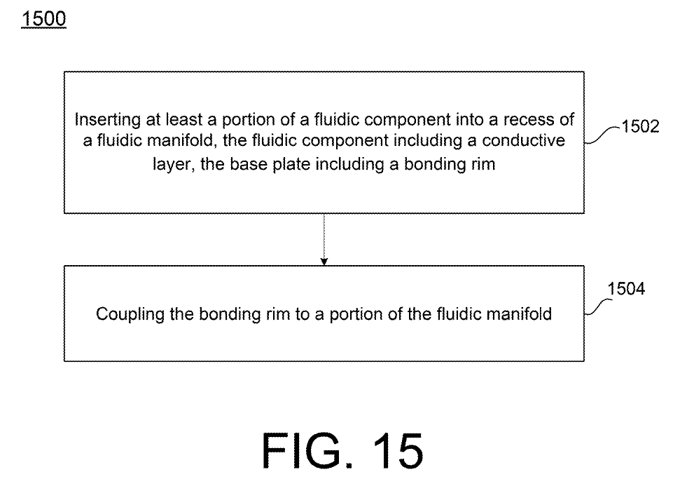

FIG. 15 illustrates a flowchart 1500 depicting example operations of coupling a fluidic component to a fluidic manifold according to an aspect. Although the flowchart 1500 of FIG. 15 illustrates the operations in sequential order, it will be appreciated that this is merely an example, and that additional or alternative operations may be included. Further, operations of FIG. 15 and related operations may be executed in a different order than that shown, or in a parallel or overlapping fashion.

Operation 1502 includes inserting at least a portion of a fluidic component into a recess of a fluidic manifold, the fluidic component including a conductive layer, the conductive layer including a bonding rim. Operation 1504 includes coupling the bonding rim to a portion of the fluidic manifold.

Clause 1. An implantable medical device comprising: an inflatable member; a fluid reservoir; and an electronic pump device configured to transfer fluid between the inflatable member and the fluid reservoir, the electronic pump device including a housing, the housing including: a fluidic manifold; and a fluidic component coupled to the fluidic manifold, the fluidic component including a conductive layer, the conductive layer including a bonding rim, the bonding rim being coupled to the fluidic manifold.

Clause 2. The implantable medical device of clause 1, wherein the bonding rim is coupled to the fluidic manifold based on a fusion weld.

Clause 3. The implantable medical device of clause 1 or 2, wherein the conductive layer includes a channel disposed around a perimeter portion of the conductive layer, the bonding rim being defined by the channel.

Clause 4. The implantable medical device of clause 3, wherein the conductive layer includes a first surface and a second surface disposed opposite to the first surface, the channel extending into the first surface.

Clause 5. The implantable medical device of clause 3, wherein the conductive layer includes a first surface and a second surface disposed opposite to the first surface, the conductive layer including an edge that extends between the first surface and the second surface, the channel extending into the channel.

Clause 6. The implantable medical device of clause 1, wherein the conductive layer includes a first surface and a second surface disposed opposite to the first surface, the bonding rim including a raised portion extending from the first surface.

Clause 7. The implantable medical device of any one of clauses 1 to 6, wherein the fluidic component includes a pump or a valve, the conductive layer including a base plate.

Clause 8. The implantable medical device of clause 7, wherein the base plate includes a first hole and a second hole.

Clause 9. The implantable medical device of any one of clauses 1 to 8, wherein the conductive layer includes a base plate, the fluidic manifold including an actuator, the fluidic manifold including a recess portion, the actuator being coupled to the base plate, the base plate being disposed within the recess portion, the actuator being coupled to a surface of the fluidic manifold.

Clause 10. An electronic pump device comprising: a fluidic manifold; and a fluidic component coupled to the fluidic manifold, the fluidic component including a conductive layer, the conductive layer including a bonding rim, the bonding rim being coupled to the fluidic manifold.

Clause 11. The electronic pump device of clause 10, wherein the bonding rim is coupled to the fluidic manifold based on a fusion weld.

Clause 12. The electronic pump device of clause 10 or 11, wherein the conductive layer includes a channel disposed around a perimeter portion of the conductive layer, the bonding rim being defined by the channel, wherein the conductive layer includes a first surface and a second surface disposed opposite to the first surface, the channel extending into the first surface.

Clause 13. A method for constructing an electronic pump device, the method comprising: inserting at least a portion of a fluidic component into a recess of a fluidic manifold, the fluidic component including a conductive layer, the conductive layer including a bonding rim; and coupling the bonding rim to a portion of the fluidic manifold.

Clause 14. The method of clause 13, wherein coupling the bonding rim to the portion of the fluidic manifold includes: creating a fusion seam by applying heat to the bonding rim and the portion of the fluidic manifold.

Clause 15. The method of clause 13 or 14, wherein the conductive layer includes a channel disposed around a perimeter portion of the conductive layer, the bonding rim being defined by the channel.

Clause 16. An implantable medical device comprising: an inflatable member; a fluid reservoir; and an electronic pump device configured to transfer fluid between the inflatable member and the fluid reservoir, the electronic pump device including a housing, the housing including: a fluidic manifold; and a fluidic component coupled to the fluidic manifold, the fluidic component including a conductive layer, the conductive layer including a bonding rim, the bonding rim being coupled to the fluidic manifold.

Clause 17. The implantable medical device of clause 1, wherein the bonding rim is coupled to the fluidic manifold based on a fusion weld.

Clause 18. The implantable medical device of clause 1, wherein the conductive layer includes a channel disposed around a perimeter portion of the conductive layer, the bonding rim being defined by the channel.

Clause 19. The implantable medical device of clause 18, wherein the conductive layer includes a first surface and a second surface disposed opposite to the first surface, the channel extending into the first surface.

Clause 20. The implantable medical device of clause 18, wherein the conductive layer includes a first surface and a second surface disposed opposite to the first surface, the conductive layer including an edge that extends between the first surface and the second surface, the channel extending into the channel.

Clause 21. The implantable medical device of clause 16, wherein the conductive layer includes a first surface and a second surface disposed opposite to the first surface, the bonding rim including a raised portion extending from the first surface.

Clause 22. The implantable medical device of clause 16, wherein the fluidic component includes a pump or a valve, the conductive layer including a base plate.

Clause 23. The implantable medical device of clause 22, wherein the base plate includes a first hole and a second hole.

Clause 24. The implantable medical device of clause 16, wherein the conductive layer includes a base plate, the fluidic manifold including an actuator, the fluidic manifold including a recess portion, the actuator being coupled to the base plate, the base plate being disposed within the recess portion, the actuator being coupled to a surface of the fluidic manifold.

Clause 25. An electronic pump device comprising: a fluidic manifold; and a fluidic component coupled to the fluidic manifold, the fluidic component including a conductive layer, the conductive layer including a bonding rim, the bonding rim being coupled to the fluidic manifold.

Clause 26. The electronic pump device of clause 25, wherein the bonding rim is coupled to the fluidic manifold based on a fusion weld.

Clause 27. The electronic pump device of clause 25, wherein the conductive layer includes a channel disposed around a perimeter portion of the conductive layer, the bonding rim being defined by the channel.

Clause 28. The electronic pump device of clause 27, wherein the conductive layer includes a first surface and a second surface disposed opposite to the first surface, the channel extending into the first surface.

Clause 29. The electronic pump device of clause 27, wherein the conductive layer includes a first surface and a second surface disposed opposite to the first surface, the conductive layer including an edge that extends between the first surface and the second surface, the channel extending into the channel.

Clause 30. The electronic pump device of clause 25, wherein the conductive layer includes a first surface and a second surface disposed opposite to the first surface, the bonding rim including a raised portion extending from the first surface.

Clause 31. The electronic pump device of clause 25, wherein the fluidic component includes a pump, a valve, or a pressure sensor.

Clause 32. The electronic pump device of clause 25, wherein the conductive layer includes a disc-shaped base plate.

Clause 33. A method for constructing an electronic pump device, the method comprising: inserting at least a portion of a fluidic component into a recess of a fluidic manifold, the fluidic component including a conductive layer, the conductive layer including a bonding rim; and coupling the bonding rim to a portion of the fluidic manifold.

Clause 34. The method of clause 33, wherein coupling the bonding rim to the portion of the fluidic manifold includes: creating a fusion seam by applying heat to the bonding rim and the portion of the fluidic manifold.

Clause 35. The method of clause 33 or 34, wherein the conductive layer includes a channel disposed around a perimeter portion of the conductive layer, the bonding rim being defined by the channel.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir; and
an electronic pump device configured to transfer fluid between the inflatable member and the fluid reservoir, the electronic pump device including a housing, the housing including:
a fluidic manifold; and
a fluidic component coupled to the fluidic manifold, the fluidic component including a conductive layer, the conductive layer including a bonding rim, the bonding rim being coupled to the fluidic manifold.

2. The implantable medical device of claim 1, wherein the bonding rim is coupled to the fluidic manifold based on a fusion weld.

3. The implantable medical device of claim 1, wherein the conductive layer includes a channel disposed around a perimeter portion of the conductive layer, the bonding rim being defined by the channel.

4. The implantable medical device of claim 3, wherein the conductive layer includes a first surface and a second surface disposed opposite to the first surface, the channel extending into the first surface.

5. The implantable medical device of claim 3, wherein the conductive layer includes a first surface and a second surface disposed opposite to the first surface, the conductive layer including an edge that extends between the first surface and the second surface, the channel extending into the channel.

6. The implantable medical device of claim 1, wherein the conductive layer includes a first surface and a second surface disposed opposite to the first surface, the bonding rim including a raised portion extending from the first surface.

7. The implantable medical device of claim 1, wherein the fluidic component includes a pump or a valve, the conductive layer including a base plate.

8. The implantable medical device of claim 7, wherein the base plate includes a first hole and a second hole.

9. The implantable medical device of claim 1, wherein the conductive layer includes a base plate, the fluidic manifold including an actuator, the fluidic manifold including a recess portion, the actuator being coupled to the base plate, the base plate being disposed within the recess portion, the actuator being coupled to a surface of the fluidic manifold.

10. An electronic pump device comprising:
a fluidic manifold; and
a fluidic component coupled to the fluidic manifold, the fluidic component including a conductive layer, the conductive layer including a bonding rim, the bonding rim being coupled to the fluidic manifold.

11. The electronic pump device of claim 10, wherein the bonding rim is coupled to the fluidic manifold based on a fusion weld.

12. The electronic pump device of claim 10, wherein the conductive layer includes a channel disposed around a perimeter portion of the conductive layer, the bonding rim being defined by the channel.

13. The electronic pump device of claim 12, wherein the conductive layer includes a first surface and a second surface disposed opposite to the first surface, the channel extending into the first surface.

14. The electronic pump device of claim 12, wherein the conductive layer includes a first surface and a second surface disposed opposite to the first surface, the conductive layer including an edge that extends between the first surface and the second surface, the channel extending into the channel.

15. The electronic pump device of claim 10, wherein the conductive layer includes a first surface and a second surface disposed opposite to the first surface, the bonding rim including a raised portion extending from the first surface.

16. The electronic pump device of claim 10, wherein the fluidic component includes a pump, a valve, or a pressure sensor.

17. The electronic pump device of claim 10, wherein the conductive layer includes a disc-shaped base plate.

18. A method for constructing an electronic pump device, the method comprising:
inserting at least a portion of a fluidic component into a recess of a fluidic manifold, the fluidic component including a conductive layer, the conductive layer including a bonding rim; and
coupling the bonding rim to a portion of the fluidic manifold.

19. The method of claim 18, wherein coupling the bonding rim to the portion of the fluidic manifold includes:
creating a fusion seam by applying heat to the bonding rim and the portion of the fluidic manifold.

20. The method of claim 18, wherein the conductive layer includes a channel disposed around a perimeter portion of the conductive layer, the bonding rim being defined by the channel.
